(19) United States
(12) Patent Application Publication
(10) Pub. No.: US 2022/0268768 A1
(43) Pub. Date: Aug. 25, 2022

(54) STRUCTURE AND METHODS FOR DETECTION OF SAMPLE ANALYTES

(71) Applicant: Palamedrix, Inc., La Jolla, CA (US)

(72) Inventors: Ashwin Gopinath, Palo Alto, CA (US); Paul Rothemund, Pasadena, CA (US); Rishabh Shetty, San Diego, CA (US); Shane Bowen, Encinitas, CA (US); Rachel Galimidi, Encinitas, CA (US)

(21) Appl. No.: 17/677,255

(22) Filed: Feb. 22, 2022

Related U.S. Application Data
(60) Provisional application No. 63/152,607, filed on Feb. 23, 2021.

(51) Int. Cl. G01N 33/543; G01N 33/58; B01L 3/00

(52) U.S. Cl. CPC G01N 33/54353; G01N 33/58; B01L 3/5085; B01L 2200/027; B01L 2200/10; B01L 2200/16; B01L 2300/0829; B01L 2300/163

(57) ABSTRACT

Provided herein are structures and methods for detecting one or more analyte molecules present in a sample. In some embodiments, the one or more analyte molecules are detected using one or more supramolecular structures. In some embodiments, the supramolecular structures facilitate binding of a single detector molecule. In some embodiments, the stable state supramolecular structures are configured to provide a signal for analyte molecule detection and quantification. In some embodiments, the signal correlates to a DNA signal, such that detection and quantification of an analyte molecule comprises converting the presence of the analyte molecule into a DNA signal.

### CROSS-REFERENCE TO RELATED APPLICATIONS

The present application claims priority to and the benefit of U.S. Provisional Application No. 63/152,607, filed Feb. 23, 2021, and entitled STRUCTURE AND METHODS FOR DETECTION OF SAMPLE ANALYTES, the disclosure of which is hereby incorporated by reference herein in its entirety.

### BACKGROUND

The current state of personalized healthcare is overwhelmingly genome-centric, predominantly focused on quantifying the genes present within an individual. While such an approach has proven to be extremely powerful, it does not provide a clinician with the complete picture of an individual's health. This is because genes are the “blueprints” of an individual and it merely informs the likelihood of developing an ailment. Within an individual these “blueprints” first need to be transcribed into RNA and then translated into various protein molecules, the real “actors” in the cell, in order to have any effect on the health of an individual.

The concentration of proteins, the interaction between the proteins (protein-protein interactions or PPI), as well as the interaction between proteins and other molecules, are intricately linked to the health of different organs, homeostatic regulatory mechanism as well as the interaction of these systems with the external environment. Hence, quantitative information about proteins and protein interactions such as PPIs is vital to create a complete picture of an individual's health at a given time point as well as to predict any emerging health issues. For instance, the amount of stress experienced by cardiac muscles (e.g. during a heart attack) can be inferred by measuring the concentration of troponin I/II and myosin light chain present within peripheral blood. Similar protein biomarkers have also been identified, validated and are deployed for a wide variety of organ dysfunctions (e.g. liver disease and thyroid disorders), specific cancers (e.g. colorectal or prostate cancer), and infectious diseases (e.g. HIV and Zika). The interaction between these proteins are also essential for drug development and are increasingly becoming a highly sought-after dataset. The ability to detect and quantify proteins and protein interaction with other molecules within a given sample of bodily fluids is an integral component of such healthcare development.

### SUMMARY

The present disclosure generally relates to systems, structures and methods for detection and quantification of analyte molecules in a sample.

Provided herein, in some embodiments, is a method for detecting an analyte molecule present in a sample, the method comprising: a) providing a supramolecular structure comprising: a core structure comprising a plurality of core molecules and a capture molecule linked to the core structure at a first location, b) contacting the sample with the supramolecular structure and c) providing a detector molecule assembly; and d) detecting the analyte molecule based on a signal provided by the detector molecule assembly and an associated signal provided by the supramolecular structure.

In some embodiments, any method disclosed herein further comprising quantifying the concentration of the analyte molecule in the sample. In some embodiments, any method disclosed herein further comprising identifying the detected analyte molecule. In some embodiments, any method disclosed herein further comprising detecting the analyte molecule based on the signal when the analyte molecule is present in the sample at a count of a single molecule or higher. In some embodiments, for any method disclosed herein, the sample comprises a complex biological sample and the method provides for single-molecule sensitivity thereby increasing a dynamic range and quantitative capture of a range of molecular concentrations within the complex biological sample. In some embodiments, for any method disclosed herein, the analyte molecule comprises a protein, a peptide, a peptide fragment, a lipid, a DNA, a RNA, an organic molecule, an inorganic molecule, complexes thereof, or any combinations thereof. In some embodiments, for any method disclosed herein, each supramolecular structure is a nanostructure.

In some embodiments, for any method disclosed herein, each core structure is a nanostructure. In some embodiments, for any method disclosed herein, the plurality of core molecules for each core structure are arranged into a pre-defined shape and/or have a prescribed molecular weight. In some embodiments, the pre-defined shape is configured to limit or prevent cross-reactivity with another supramolecular structure. In some embodiments, for any method disclosed herein, the plurality of core molecules for each core structure comprises one or more nucleic acid strands, one or more branched nucleic acids, one or more peptides, one or more small molecules, or combinations thereof. In some embodiments, for any method disclosed herein, each core structure independently comprises a scaffolded deoxyribonucleic acid (DNA) origami, a scaffolded ribonucleic acid (RNA) origami, a scaffolded hybrid DNA:RNA origami, a single-stranded DNA tile structure, a multi-stranded DNA tile structure, a single-stranded RNA origami, a multi-stranded RNA tile structure, hierarchically composed DNA or RNA origami with multiple scaffolds, a peptide structure, or combinations thereof.

In some embodiments, for any method disclosed herein, the respective analyte molecule is 1) bound to the capture molecule of the respective supramolecular structure through a chemical bond and/or 2) bound to the detector molecule of the detector molecule assembly through a chemical bond. In some embodiments, for any method disclosed herein, the capture molecule and detector molecule independently comprise a protein, a peptide, an antibody, an aptamer (RNA and DNA), a fluorophore, a darpin, a catalyst, a polymerization initiator, a polymer like PEG, or combinations thereof. In some embodiments, for any method disclosed herein, wherein for each supramolecular structure: a) the capture molecule is linked to the core structure through a capture barcode, wherein the capture barcode comprises a first capture linker, a second capture linker, and a capture bridge disposed between the first and second capture linkers, wherein the first capture linker is bound to a first core linker that is bound to the first location on the core structure, wherein the capture molecule and the second capture linker are linked together through binding to a third capture linker, and b) the detector molecule assembly includes a detector barcode, wherein the detector barcode comprises one or more linkers. In some embodiments, the capture bridge and detector molecule assembly independently comprise a polymer core. In some embodiments, the polymer core of the capture bridge and the polymer core of the detector molecule assembly independently comprise a nucleic acid (DNA or RNA) of specific sequence or a polymer like PEG.

In some embodiments, for any method disclosed herein, each supramolecular structure further comprises an anchor molecule linked to the core structure. In some embodiments, the anchor molecule is linked to the core structure via an anchor barcode, wherein the anchor barcode comprises a first anchor linker, a second anchor linker, and an anchor bridge disposed between the first and second anchor linkers, wherein the first anchor linker is bound to a third core linker that is bound to a third location on the core structure, wherein the anchor molecule is linked to the second anchor linker. In some embodiments, the anchor molecule comprises an amine, a thiol, a DBCO, a maleimide, biotin, an azide, an acrydite, a NHS-ester, a single stranded nucleic acid (RNA or DNA) of specific sequence, one or more polymers like PEG or polymerization initiators, or combinations thereof. In some embodiments, the anchor bridge comprises a polymer core. In some embodiments, the polymer core of the anchor bridge comprises a nucleic acid (DNA or RNA) of specific sequence or a polymer like PEG. In some embodiments, the third core linker, first anchor linker, second anchor linker, and anchor molecule independently comprise an anchor reactive molecule or DNA sequence domain. In some embodiments, each anchor reactive molecule independently comprises an amine, a thiol, a DBCO, a maleimide, biotin, an azide, an acrydite, a NHS-ester, a single stranded nucleic acid (RNA or DNA) of specific sequence, one or more polymers like PEG or polymerization initiators, or combinations thereof. In some embodiments, the anchor molecule is linked to the second anchor linker through a chemical bond. In some embodiments, the anchor molecule is covalently bonded to the second anchor linker.

In some embodiments, for any method disclosed herein, the signal comprises the detector barcode, the capture barcode, or combinations thereof, corresponding to a supramolecular structure that is bound to an analyte that in turn is bound to a detector molecule assembly. In some embodiments, each detector barcode provides a DNA signal corresponding to the detector molecule and providing information indicative of its specificity for an analyte molecule bound to the respective detector molecule. In some embodiments, the detector barcodes are analyzed using genotyping, qPCR, sequencing, or combinations thereof. In some embodiments, a plurality of analyte molecules in the sample are detected simultaneously through multiplexing. In some embodiments, for any method disclosed herein, the capture and detector molecules for each supramolecular structure is configured for binding to one or more specific types of analyte molecules.

In some embodiments, for any method comprising using a plurality of supramolecular structures disclosed herein, each core structure of the plurality of supramolecular structures are identical to each other. In some embodiments, each supramolecular structure comprises a prescribed shape, size, molecular weight, or combinations thereof, so as to reduce or eliminate cross-reactions between a plurality of supramolecular structures. In some embodiments, each supramolecular structure comprises a plurality of capture molecules. In some embodiments, each supramolecular structure comprises a prescribed stoichiometry of the capture and detector molecules so as to reduce or eliminate cross-reactions between the plurality of supramolecular structures.

In some embodiments, one or more supramolecular structures are attached to a hydrogelporous matrix. In some embodiments, each supramolecular structure is co-polymerized with the hydrogel through a corresponding anchor molecule linked to the respective core structure of the corresponding supramolecular structure. In some embodiments, the one or more supramolecular structures are embedded within the hydrogel. In some embodiments, a plurality of supramolecular structures are disposed on a substrate, such as a shaped or planar substrate, wherein the substrate comprises a plurality of binding sites, wherein each binding site is configured to link with a corresponding supramolecular structure. In some embodiments, the plurality of supramolecular structures are configured to detect the same analyte molecule. In some embodiments, for any method comprising using a substrate, the method further comprises providing a plurality of signaling elements linked with the detector molecules. In some embodiments, each signaling element comprises a fluorescent molecule or microbead, a fluorescent polymer, highly charged nanoparticles or polymer. In some embodiments, at least one supramolecular structure of the plurality of supramolecular structures is configured to detect a different analyte molecule from the other supramolecular structures.

In some embodiments, for any method comprising using a planar substrate, further comprising barcoding each supramolecular structure so as to identify the location of each supramolecular structure on the planar substrate. In some embodiments, for any method comprising using a planar substrate, the method comprises providing a plurality of signaling elements as provided herein that are configured to link with the detector molecules.

In some embodiments, for any method disclosed herein, the sample comprises a biological particle or a biomolecule. In some embodiments, for any method disclosed herein, the sample comprises an aqueous solution comprising a protein, a peptide, a fragment of a peptide, a lipid, DNA, RNA, an organic molecule, a viral particle, an exosome, an organelle, or any complexes thereof. In some embodiments, for any method disclosed herein, the sample comprises a tissue biopsy, blood, blood plasma, Urine, Saliva, Tear, Cerebrospinal fluid, extracellular fluid, cultures cells, culture media, discarded tissue, plant matter, a synthetic protein, prions, a bacterial and/or viral sample or fungal tissue, or combinations thereof. The sample may be processed to release the analytes from cells or to otherwise prepare the sample for analysis prior to contacting the sample with the supramolecular structures provided herein. The sample may be an environmental sample, such as a wastewater or soil sample. The sample may also be a nonbiological sample. In an embodiment, the sample may be a sample from a chemical process step, a sample of food or nutritional components, or packaging components.

Provided herein, in some embodiments, is a substrate for detecting one or more analyte molecules in a sample, the substrate comprising a plurality of supramolecular structures, each supramolecular structure comprising: a) a core structure comprising a plurality of core molecules, and b) a capture molecule linked to the supramolecular core.

In some embodiments, each core structure of the plurality of supramolecular structures is identical to each other. In some embodiments, the substrate comprises a solid support, solid substrate, a polymer matrix, or a molecular condensate. In some embodiments, the sample comprises a complex biological sample and the method provides for single-molecule sensitivity thereby increasing a dynamic range and quantitative capture of a range of molecular concentrations within the complex biological sample. In some embodiments, the one or more analyte molecules comprises a protein, a peptide, a peptide fragment, a lipid, a DNA, a RNA, an organic molecule, an inorganic molecule, complexes thereof, or any combinations thereof. In some embodiments, each supramolecular structure is a nanostructure. In some embodiments, each core structure is a nanostructure. In some embodiments, the plurality of core molecules for each core structure are arranged into a pre-defined shape and/or have a prescribed molecular weight. In some embodiments, the pre-defined shape is configured to limit or prevent cross-reactivity with another supramolecular structure. In some embodiments, the plurality of core molecules for each core structure comprises one or more nucleic acid strands, one or more branched nucleic acids, one or more peptides, one or more small molecules, or combinations thereof. In some embodiments, each core structure independently comprises a scaffolded deoxyribonucleic acid (DNA) origami, a scaffolded ribonucleic acid (RNA) origami, a scaffolded hybrid DNA:RNA origami, a single-stranded DNA tile structure, a multi-stranded DNA tile structure, a single-stranded RNA origami, a multi-stranded RNA tile structure, hierarchically composed DNA or RNA origami with multiple scaffolds, a peptide structure, or combinations thereof. In some embodiments, the respective analyte molecule is 1) bound to the capture molecule through a chemical bond and/or 2) bound to the detector molecule through a chemical bond. In some embodiments, the capture molecule and detector molecule independently comprise a protein, a peptide, an antibody, an aptamer (RNA and DNA), a fluorophore, a darpin, a catalyst, a polymerization initiator, a polymer like PEG, or combinations thereof.

In some embodiments, wherein for each supramolecular structure of the substrate: a) the capture molecule is linked to the core structure through a capture barcode, wherein the capture barcode comprises a first capture linker, a second capture linker, and a capture bridge disposed between the first and second capture linkers, wherein the first capture linker is bound to a first core linker that is bound to the first location on the core structure, wherein the capture molecule and the second capture linker are linked together through binding to a third capture linker. The capture molecule binds to an analyte that in turn binds to a detector molecule of a detector molecule assembly. In an embodiment, the detector molecule assembly includes a separate supramolecular structure that is not linked to the capture molecule and that is not immobilized on the substrate.

In some embodiments, the supramolecular structure that includes the capture molecule directly interacts with a substrate material to immobilize the supramolecular structure on the substrate. In some embodiments, the supramolecular structure that includes the capture molecule further comprises an anchor molecule, as provided herein, linked to the core structure, and the anchor molecule is linked to the substrate to immobilize the supramolecular structure on the substrate.

In some embodiments, the signal read or detected from the substrate comprises the detector barcode, the capture barcode, or combinations thereof that may be analyzed using optical sensors, magnetic sensors, and/or electrical sensors. Detection techniques include electrochemical sensing, genotyping, qPCR, sequencing, or combinations thereof. In some embodiments, one or more supramolecular structures are configured for multiplexing the sample, wherein a plurality of analyte molecules in the sample are detected simultaneously. In some embodiments, the capture molecules for each supramolecular structure are configured for binding to one or more specific types of analyte molecules.

In some embodiments, the sample comprises a complex biological sample and the method provides for single-molecule sensitivity thereby increasing a dynamic range and quantitative capture of a range of molecular concentrations within the complex biological sample. In some embodiments, the analyte molecule comprises a protein, a peptide, a peptide fragment, a lipid, a DNA, a RNA, an organic molecule, an inorganic molecule, complexes thereof, or any combinations thereof. In some embodiments, the supramolecular structure is a nanostructure. In some embodiments, the core structure is a nanostructure. In some embodiments, the supramolecular structure comprises a prescribed shape, size, molecular weight, or combinations thereof, so as to reduce or eliminate cross-reactions with another supramolecular structure. In some embodiments, the supramolecular structure comprises a plurality of capture molecules.

In some embodiments, the plurality of core molecules for the core structure are arranged into a pre-defined shape and/or have a prescribed molecular weight. In some embodiments, the pre-defined shape is configured to limit or prevent cross-reactivity with another supramolecular structure. In some embodiments, the plurality of core molecules for each core structure comprises one or more nucleic acid strands, one or more branched nucleic acids, one or more peptides, one or more small molecules, or combinations thereof. In some embodiments, the core structure independently comprises a scaffolded deoxyribonucleic acid (DNA) origami, a scaffolded ribonucleic acid (RNA) origami, a scaffolded hybrid DNA:RNA origami, a single-stranded DNA tile structure, a multi-stranded DNA tile structure, a single-stranded RNA origami, a multi-stranded RNA tile structure, hierarchically composed DNA or RNA origami with multiple scaffolds, a peptide structure, or combinations thereof.

### BRIEF DESCRIPTION OF THE DRAWINGS

Specific embodiments of the disclosed devices, delivery systems, or methods will now be described with reference to the drawings. Nothing in this detailed description is intended to imply that any particular component, feature, or step is essential to the invention.

FIG. 1 shows a supramolecular structure and the related subcomponents according to embodiments of the disclosure.

FIG. 2 shows a supramolecular structure with an associated analyte and detector molecule assembly according to embodiments of the disclosure.

FIG. 3 shows the supramolecular structure of FIG. 1 with an associated analyte and detector molecule assembly and the related subcomponents according to embodiments of the disclosure.

FIG. 4 shows a supramolecular structure with an associated analyte and detector molecule assembly that includes a detector core structure according to embodiments of the disclosure.

FIG. 5 shows specific binding of a supramolecular structure and a detector molecule assembly to an analyte according to embodiments of the disclosure.

FIG. 6 shows an analyte with no specific binding to a supramolecular structure according to embodiments of the disclosure.

FIG. 7 shows a workflow for analyte and detector binding to a supramolecular structure array according to embodiments of the disclosure.

FIG. 8 shows an example capture molecule identification used in conjunction with analyte detection according to embodiments of the disclosure.

FIG. 9 shows an example of analyte detection according to embodiments of the disclosure.

FIG. 10 shows an example of analyte detection according to embodiments of the disclosure.

FIG. 11 shows an example of a supramolecular structure array that may be used in analyte detection according to embodiments of the disclosure.

FIG. 12 shows an example of a supramolecular structure array that may be used in analyte detection according to embodiments of the disclosure.

FIG. 13 shows an example of a supramolecular structure array that may be used in analyte detection according to embodiments of the disclosure.

FIG. 14 shows an example of a supramolecular structure array that may be used in antigen detection according to embodiments of the disclosure.

FIG. 15 shows an example porous matrix supramolecular structure array that may be used in analyte detection according to embodiments of the disclosure.

FIG. 16 shows a block diagram of an example analyte detection system according to embodiments of the disclosure.

### DETAILED DESCRIPTION

Disclosed herein are structures and methods for detecting one or more analyte molecules present in a sample. In some embodiments, the one or more analyte molecules are detected based on capture by one or more supramolecular structures. In some embodiments, the one or more supramolecular structures include or are linked to a capture molecule that specifically binds to an analyte present in a sample. The bound analyte in turn interacts with a detector molecule of a detector molecule assembly that has a detectable moiety, such as a unique identifier (e.g., a nucleic acid sequence, a peptide, a polysaccharide, an acrydite) and/or a molecule that includes, interacts with, or that can be used to dock other molecules that can be detected (e.g., optically, electrically, magnetically). In some embodiments, the detector molecule assembly generates a DNA signal, such that detection and quantification of binding of an analyte molecule to the capture molecule comprises converting the presence of the analyte molecule into a DNA signal through amplification of the unique identifier of the supramolecular structure. In some embodiments, the detector molecule assembly is linked to an enzyme that converts a substrate to an optically detectable signal. In an embodiment, the supramolecular structure is a nucleic acid origami that is linked to or immobilized on a substrate. In an embodiment, the supramolecular structure carries a capture molecule via a barcode that include the unique identifier for the capture molecule and that links the capture molecule to a scaffold of the supramolecular structure.

In some embodiments, the disclosed techniques provide a single molecule enzyme-linked immunosorbent assay (ELISA) in which the supramolecular structure and associated capture molecule operate as a capture entity and the detector molecule assembly operates as the detection entity that is used to generate a detection signal (e.g., when reacted with appropriate detection reagents that are sensed using sensors of a detection system). Use of the supramolecular structure as the capture entity permits specific identification and, in embodiments, location mapping of each individual capture molecule immobilized on a substrate. Further, the supramolecular structures are configured to be organized on a substrate or within a porous material to permit single molecule binding.

Thus, as provided herein, detectable analyte binding can be associated with an individual capture molecule among many different capture molecules on the substrate to generate assay results in which binding characteristics of an analyte pool of multiple different analytes are characterized. This in turn permits a sample having an uncharacterized composition of analytes to analyzed for the presence and/or concentration of particular analytes of interest. For example, a human sample can be characterized to determine a presence and/or concentration of antibodies with binding specificity to particular antigens in a panel of antigen capture molecules, such that the capture molecules represent a known infectious disease antigen panel. The assay results may show positive binding results associated with a particular antigen, which is indicative of the presence of antibodies in the subject providing the sample. In another embodiment, the identity of analytes in the sample may be at least partially known, but their binding affinity may not be characterized for a particular pool of capture molecules. For example, the capture molecules can be a set of candidate drugs, and the analytes can be molecules in human blood. Binding of a drug candidate to such a protein can be used to assess bioavailability or potential off-target binding. The assay results may show positive binding results associated with a particular drug candidate that can in turn be mapped to a particular analyte, which is based on identification of particular detector binding (e.g., identifying binding by barcode identification in a detector molecule assembly that includes an antibody specific for the analyte).

While conventional ELISA protocols may include detectable fluorescent signals generated by enzymes linked to detection antibodies as an indicator of binding, the disclosed techniques may additionally or alternatively provide amplified nucleic acid signals from a unique identifier of the detector molecule assembly and from which sequence information can be determined or from which an optically detectable signal is released that corresponds to amplification (e.g., qPCR using a primer/probe set specific for the unique identifier). Thus, unique identity information for the detector molecule assembly permits specific identification of the particular detector molecule that is linked to a capture molecule via the bound analyte in certain embodiments. However, in embodiments, the detector molecule assembly may not carry a unique identifier. Other detection techniques may include optical, magnetic, and or electrical detection techniques

### Sample

Disclosed embodiments relate to analyte detection in which the analytes are present in a sample, such as a biological sample. In some embodiments, the sample comprises an aqueous solution comprising protein, peptides, peptide fragments, lipids, DNA, RNA, organic molecules, inorganic molecules, complexes thereof, or any combinations thereof. In some embodiments, the analyte molecules in the sample comprise protein, peptides, peptide fragments, lipids, DNA, RNA, organic molecules, inorganic molecules, complexes thereof, or any combinations thereof. In some embodiments, the analyte molecules comprise comprises intact proteins, denatured proteins, partially or fully degraded proteins, peptide fragments, denatured nucleic acids, degraded nucleic acid fragments, complexes thereof, or combinations thereof. In some embodiments, the sample is obtained from tissue, cells, the environment of tissues and/or cells, or combinations thereof. In some embodiments, the sample comprises tissue biopsy, blood, blood plasma, urine, saliva, a tear, cerebrospinal fluid, extracellular fluid, cultures cells, culture media, discarded tissue, plant matter, synthetic proteins, bacterial, viral samples, fungal tissue, or combinations thereof. In some embodiments, the sample is isolated from a primary source such as cells, tissue, bodily fluids (e.g., blood), environmental samples, or combinations thereof, with or without purification. In some embodiments, the cells are lysed using a mechanical process or other cell lysis methods (e.g., lysis buffer). In some embodiments, the sample is filtered using a mechanical process (e.g., centrifugation), micron filtration, chromatography columns, other filtration methods, or combinations thereof. In some embodiments, the sample is treated with one or more enzymes to remove one or more nucleic acids or one or more proteins. In some embodiments, the sample comprises intact proteins, denatured proteins, partially or fully degraded proteins, peptide fragments, denatured nucleic acids or degraded nucleic acid fragments. In some embodiments, the sample is collected from one or more individual persons, one or more animals, one or more plants, or combinations thereof. In some embodiments, the sample is collected from an individual person, animal and/or plant having a disease or disorder that comprises an infectious disease, an immune disorder, a cancer, a genetic disease, a degenerative disease, a lifestyle disease, an injury, a rare disease, an age-related disease, or combinations thereof.

### Supramolecular Structure

In some embodiments, the supramolecular structure is a programmable structure that can spatially organize molecules. In some embodiments, the supramolecular structure comprises a plurality of molecules linked together. In some embodiments, the plurality of molecules of the supramolecular structure interact with at least some of each other. In some embodiments, the supramolecular structure comprises a specific shape. In some embodiments, the supramolecular nanostructure comprises a prescribed molecular weight based on the plurality of molecules of the supramolecular structure. In some embodiments, the supramolecular structure is a nanostructure. In some embodiments the plurality of molecules are linked together through a bond, a chemical bond, a physical attachment, or combinations thereof. In some embodiments, the supramolecular structure comprises a large molecular entity, of specific shape and molecular weight, formed from a well-defined number of smaller molecules interacting specifically with each other. In some embodiments, the structural, chemical, and physical properties of the supramolecular structure are explicitly designed. In some embodiments, the supramolecular structure comprises a plurality of subcomponents that are spaced apart according to a prescribed distance. In some embodiments, at least a portion of the supramolecular structure is rigid. In some embodiments, at least a portion of the supramolecular structure is semi-rigid. In some embodiments, at least a portion of the supramolecular structure is flexible.

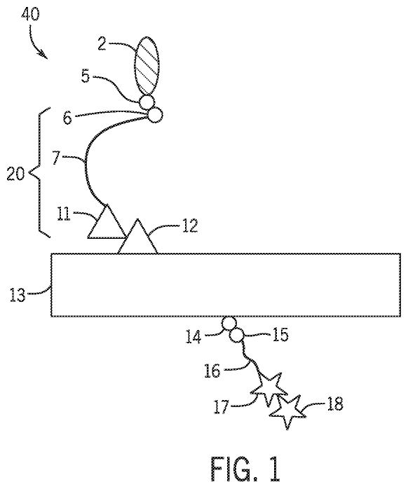

FIG. 1 provides an exemplary embodiment of a supramolecular structure 40 comprising a core structure 13, a capture molecule 2, and an anchor molecule 18. In some embodiments, the supramolecular structure comprises one or more capture molecules 2and, optionally, one or more anchor molecules 18. In some embodiments, the supramolecular structure does not comprise an anchor molecule. In some embodiments, the supramolecular structure is a polynucleotide structure.

In some embodiments, the core structure 13 comprises one or more core molecules linked together. In some embodiments, the one or more core molecules comprise 2, 3, 4, 5, 6, 7, 8, 9, 10, 20, 50, 100, 200 or 500 unique molecules that are linked together. In some embodiments, the one or more core molecules comprises from about 2 unique molecules to about 1000 unique molecules. In some embodiments, the one or more core molecules interact with each other and define the specific shape of the supramolecular structure. In some embodiments, the plurality of core molecules interact with each other through reversible non-covalent interactions.

In some embodiments, the specific shape of the core structure is a three-dimensional (3D) configuration. In some embodiments, the one or more core molecules provide a specific molecular weight. In some embodiments, the core structure 13 is a nanostructure. In some cases, the one or more core molecules comprise one or more nucleic acid strands (e.g., DNA, RNA, unnatural nucleic acids), one or more branched nucleic acids, one or more peptides, one or more small molecules, or combinations thereof. In some embodiments, the core structure comprises a polynucleotide structure. In some embodiments, at least a portion of the core structure is rigid. In some embodiments, at least a portion of the core structure is semi-rigid. In some embodiments, at least a portion of the core structure is flexible. In some embodiments, the core structure comprises a scaffolded deoxyribonucleic acid (DNA) origami, a scaffolded ribonucleic acid (RNA) origami, a scaffolded hybrid DNA/RNA origami, a single-stranded DNA tile structure, a multi-stranded DNA tile structure, a single-stranded DNA origami, a single-stranded RNA origami, a single-stranded RNA tile structure, a multi-stranded RNA tile structures, a hierarchically composed DNA and/or RNA origami with multiple scaffolds, a peptide structure, or combinations thereof. In some embodiments, the DNA origami is scaffolded. In some embodiments, the RNA origami is scaffolded. In some embodiments, the hybrid DNA/RNA origami is scaffolded. In some embodiments, the core structure comprising a DNA origami, RNA origami, or hybrid DNA/RNA origami that comprises a prescribed two-dimensional (2D) or 3D shape.

In an embodiment, the nucleic acid origami has at least one lateral dimension between about 50 nm to about 1 μm. In an embodiment, the nucleic acid origami has at least one lateral dimension between about 50 nm to about 200 nm, about 50 nm to about 400 nm, about 50 nm to about 600 nm, about 50 nm to about 800 nm, about 100 nm to about 200 nm, about 100 nm to about 300 nm, about 100 nm to about 400 nm, about 100 nm to about 500 nm, about 200 nm to about 400 nm by way of example. In an embodiment, the nucleic acid origami has at least a first lateral dimension between about 50 nm to about 1 μ and a second lateral dimension, orthogonal to the first, between about 50 nm to about 1 μ. In an embodiment, the nucleic acid origami has a planar footprint having an area of about 200 nm² to about 1 μ².

As shown in FIG. 1, in some embodiments, the core structure 13 is configured to be linked to a capture molecule 2an anchor molecule 18, or combinations thereof. In some embodiments, the capture molecule 2and/or anchor molecule 18 are immobilized with respect to the core nanostructure 13 when linked thereto. In some embodiments, any number of the one or more core molecules comprises one or more core linkers 12, 14 configured to form a linkage with a capture molecule 2 and/or an anchor molecule 18. In some embodiments, any number of the one or more core molecules are configured to be linked with one or more core linkers 12,14 that are configured to form a linkage with a capture molecule 2 and/or an anchor molecule 18. In some embodiments, one or more core linkers are linked to one or more core molecules through a chemical bond. In some embodiments, at least one of the one or more core linkers comprises a core reactive molecule. In some embodiments, each core reactive molecule independently comprises an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, at least one of the one or more core linkers comprises a DNA sequence domain.

In some embodiments, the core structure 13 is linked to 1) a capture molecule 2 at a prescribed location on the core structure, and optionally 2) an anchor molecule 18 at a prescribed different location on the core structure.

In some embodiments, a specified first core linker 12 is disposed at the first location on the core structure. In some embodiments, one or more core molecules at the first location are modified to form a linkage with the first core linker 12. In some embodiments, the first core linker 12 is an extension of the core structure 13.

In some embodiments, a specified third core linker 14 is disposed at the third location on the core structure 13. In some embodiments, one or more core molecules at the third location is modified to form a linkage with the third core linker 14. In some embodiments, the third core linker 14 is an extension of the core structure 13. In some embodiments, the first and second locations are disposed on a first side of the core structure 13, and the optional third location is disposed on a second side of the core structure 13.

In some embodiments, the capture molecule 2 comprises a protein, a peptide, an antibody, an aptamers (RNA and DNA), a fluorophore, a nanobody, a darpin, a catalyst, a polymerization initiator, a polymer like PEG, an organic molecule, or combinations thereof. In some embodiments, the anchor molecule comprises a reactive molecule. In some embodiments, the anchor molecule 18 comprises a reactive molecule. In some embodiments, the anchor molecule 18 comprises a DNA strand comprising a reactive molecule. In some embodiments, the anchor molecule 18 comprises an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the anchor molecule 18 comprises a protein, a peptide, an antibody, an aptamers (RNA and DNA), a fluorophore, a nanobody, a darpin, a catalyst, a polymerization initiator, a polymer like PEG, an organic molecule or combinations thereof. In some embodiments, a single capture molecule 2 is linked to the core structure 13. In some embodiments, a plurality of capture molecules 2 are linked to a core structure 13. In some embodiments, the plurality of pairs of capture molecules 2 are spaced apart from each other to minimize cross-talk. For example, different capture molecules 2 on a same core structure 13 may represent different binding sites for a same analyte molecule or may bind different analyte molecules. In another example, multiple same capture molecules 2 may be present on a core structure 13.

In some embodiments, each component of the supramolecular structure may be independently modified or tuned. In some embodiments, modifying one or more of the components of the supramolecular structure may modify the 2D and 3D geometry of the supramolecular structure itself. In some embodiments, modifying one or more of the components of the supramolecular structure may modify the 2D and 3D geometry of the core structure. In some embodiments, such capability for independently modifying the components of the supramolecular nanostructure enables precise control over the organization of one or more supramolecular structures on solid surfaces (e.g., planar surfaces or microparticles) and 3D volumes (e.g., within a hydrogel matrix).

### Capture Barcode

As shown in FIG. 1, in some embodiments, the capture molecule 2 is linked to the core structure 13 through a capture barcode 20. In some embodiments, the capture barcode 20 forms a linkage with the capture molecule 2, and the capture barcode 20 forms a linkage with the core structure 13. In some embodiments, the capture barcode 20 comprises a first capture linker 11, a second capture linker 6, and a capture bridge 7. In some embodiments, the first capture linker 11 comprises a reactive molecule. In some embodiments, the first capture linker 11 comprises a reactive molecule comprising an amine, a thiol, a DBCO, a NHS ester, a maleimide, an azide, an acrydite, a single stranded nucleic acid (e.g., RNA or DNA) of specific sequence, or a polymer (e.g., polyethylene glycol (PEG) or one or more polymerization initiators). In some embodiments, the first capture linker 11 comprises a DNA sequence domain. In some embodiments, the second capture linker 6 comprises a reactive molecule. In some embodiments, the second capture linker 6 comprises a reactive molecule comprising an amine, a thiol, a DBCO, a NHS ester, biotin, a maleimide, an azide, an acrydite, a single stranded nucleic acid (e.g., RNA or DNA) of specific sequence, or a polymer (e.g., polyethylene glycol (PEG) or one or more polymerization initiators). In some embodiments, the second capture linker comprises a DNA sequence domain. In some embodiments, the capture bridge 7 comprises a polymer. In some embodiments, the capture bridge 7 comprises a polymer that comprises a nucleic acid (e.g., DNA or RNA) of a specific sequence. In some embodiments, the capture bridge 7 comprises a polymer such as PEG. In some embodiments, the first capture linker 11 is attached to the capture bridge 7 at a first terminal end thereof, and the second capture linker 6 is attached to the capture bridge 7 at a second terminal end thereof. In some embodiments, the first capture linker 11 is attached to the capture bridge 7 via a chemical bond. In some embodiments, the second capture linker 6 is attached to the capture bridge 7 via a chemical bond. In some embodiments, the first capture linker 11 is attached to the capture bridge 7 via a physical attachment. In some embodiments, the second capture linker 6 is attached to the capture bridge 7 via a physical attachment.

In some embodiments, the capture barcode 20 is linked to the core structure 13 through a linkage between the first capture linker 11 and the first core linker 12. In some embodiments, as described herein, the first core linker 12 is disposed at a first location on the core structure 13. In some embodiments, the first capture linker 11 and first core linker 12 are linked together through a chemical bond. In some embodiments, the first capture linker 11 and first core linker 12 are linked together through a covalent bond.

In some embodiments, the capture barcode 20 is linked to the capture molecule 2 through a linkage between the second capture linker 6 and a third capture linker 5 that is bound to the capture molecule 2. In some embodiments, the third capture linker 5 comprises a reactive molecule. In some embodiments, the third capture linker 5 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the third capture linker 5 comprises a DNA sequence domain. In some embodiments, the capture molecule 2 is bound to the third capture linker 5 through a chemical bond. In some embodiments, the capture molecule 2 is bound to the third capture linker 5 through a covalent bond. In some embodiments, the second capture linker 6 and third capture linker 5 are linked together through a chemical bond. In some embodiments, the second linker 6 and third capture linker 5 are linked together through a covalent bond.

### Anchor Barcode

As shown in FIG. 1, in some embodiments, the anchor molecule 18 is linked to the core structure 13 through an anchor barcode. In some embodiments, the anchor barcode forms a linkage with the anchor molecule 18, and the anchor barcode forms a linkage with the core structure 13. In some embodiments, the anchor barcode comprises a first anchor linker 15, a second anchor linker 17, and an anchor bridge 16. In some embodiments, the first anchor linker 15 comprises a reactive molecule. In some embodiments, the first anchor linker 15 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the first anchor linker 15 comprises a DNA sequence domain. In some embodiments, the second anchor linker 17 comprises a reactive molecule. In some embodiments, the second anchor linker 17 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the second anchor linker 17 comprises a DNA sequence domain. In some embodiments, the anchor bridge 16 comprises a polymer. In some embodiments, the anchor bridge 16 comprises a polymer that comprises a nucleic acid (DNA or RNA) of a specific sequence. In some embodiments, the anchor bridge 16 comprises a polymer such as PEG. In some embodiments, the first anchor linker 15 is attached to the anchor bridge 16 at a first terminal end thereof, and the second anchor linker 17 is attached to the anchor bridge 16 at a second terminal end thereof. In some embodiments, the first anchor linker 15 is attached to the anchor bridge 16 via a chemical bond. In some embodiments, the second anchor linker 17 is attached to the anchor bridge 16 via a physical attachment. In some embodiments, the first anchor linker 15 is attached to the anchor bridge 16 via a chemical bond. In some embodiments, the second anchor linker 17 is attached to the anchor bridge 16 via a physical attachment.

In some embodiments, the anchor barcode is linked to the core structure 13 through a linkage between the first anchor linker 15 and the third core linker 14. In some embodiments, as described herein, the third core linker 14 is disposed at a third location on the core structure 13. In some embodiments, the first anchor linker 15 and third core linker 14 are linked together through a chemical bond. In some embodiments, the first anchor linker 15 and third core linker 14 are linked together through a covalent bond.

In some embodiments, the anchor barcode is linked to the anchor molecule 18 through a linkage between the second anchor linker 17 and the anchor molecule 18. As disclosed herein, in some embodiments, the anchor molecule comprises a reactive molecule, a reactive molecule, a DNA sequence domain, a DNA sequence domain comprising a reactive molecule, or combinations thereof. In some embodiments, the anchor molecule 18 is bound to the second anchor linker 17 through a chemical bond. In some embodiments, the anchor molecule 18 is bound to the second anchor linker 17 through a covalent bond.

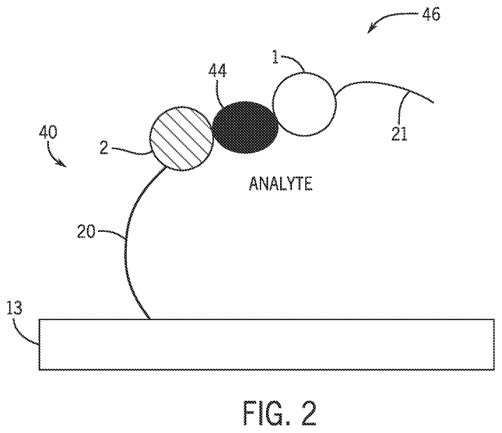

FIG. 2 is a schematic illustration of the supramolecular structure 40 bound to a corresponding analyte molecule 44 having binding specificity for the capture molecule 2. The supramolecular structure 40 is capable of binding to one or more analyte molecules 44 as a function of the particular capture molecule 2 associated with the supramolecular structure 40. The analyte molecule 44 is also capable of binding to a detector molecule assembly 46. The detector molecule assembly 46 includes a detector molecule 1 that binds with specificity to the analyte molecule 44. FIG. 2 shows a sandwich-type binding arrangement in which the capture molecule 2 and the detector molecule 1 bind to different sites on the analyte molecule 44. In some embodiments, the detector molecule 1 comprises a protein, a peptide, an antibody, an aptamers (RNA and DNA), a fluorophore, a nanobody, a darpin, a catalyst, a polymerization initiator, a polymer like PEG, an organic molecule, or combinations thereof. As shown in FIG. 2, in some embodiments, the detector molecule 1 is linked to a detector barcode 21. In some embodiments, the detector barcode 21 forms a linkage with the detector molecule 1, and may include one or more intervening components. In other embodiments, the detector molecule 1 is linked to a detectable tail or linker, but does not carry unique barcode information.

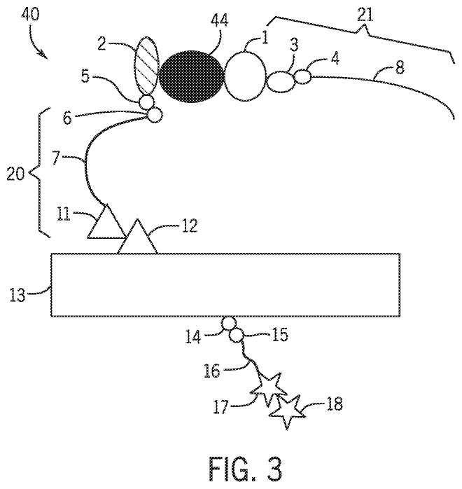

FIG. 3 shows an example arrangement of the detector barcode 21. In some embodiments, the detector barcode comprises one or more detector linkers including a first detector linker 4. The detector barcode 21 may include a dock 8 that serves as an attachment or extension/amplification site to facilitate detection. In some embodiments, the linker 4 comprises a reactive molecule. In some embodiments, the linker 4 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the linker 4 comprises a DNA sequence domain. In some embodiments, the dock 8 comprises a polymer. In some embodiments, the dock 8 comprises a polymer that comprises a nucleic acid (DNA or RNA) of a specific sequence, e.g., a single-stranded or double-stranded nucleic acid. In some embodiments, the dock 8 comprises a polymer such as PEG. In some embodiments, the linker 4 is attached to the dock 8 at a terminal end thereof, and another detector linker 4 is attached to the dock 8 at a second terminal end thereof. The attachments may be via a chemical bond or a physical attachment.

In some embodiments, the detector barcode 21 is linked to the detector molecule 1 through a linkage between a plurality of linkers, shown here as the detector linker 4 and a second detector linker 3 bound to the detector molecule 1. In some embodiments, the second detector linker 3 comprises a reactive molecule. In some embodiments, the second detector linker 3 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the second detector linker 3 comprises a DNA sequence domain. In some embodiments, the detector molecule 1 is bound to the second detector linker 3 through a chemical bond. In some embodiments, the detector molecule 1 is bound to the second detector linker 3 through a covalent bond. In some embodiments, the second detector linker 4 and second detector linker 3 are linked together through a chemical bond. In some embodiments, the second detector linker 4 and second detector linker 3 are linked together through a covalent bond.

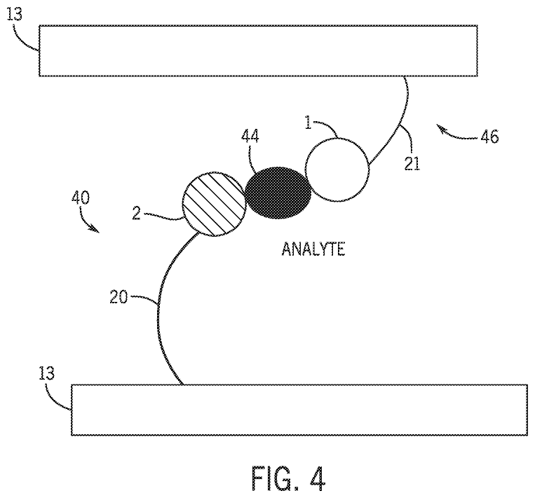

As provided herein, a capture molecule assembly includes a supramolecular structure 40 having the capture molecule 1 and the core structure 13. In certain embodiments, the detector molecule assembly 46 is linked to or coupled to a core structure 13, such that the detector molecule assembly is a supramolecular structure as provided herein as well. Thus, as shown in FIG. 4, detector molecule binding to the captured analyte molecule 44 associated with the capture molecule assembly supramolecular structure 40 creates a supramolecular structure sandwich. Generally, only one of the supramolecular structure 40 of the capture molecule assembly or the detector molecule assembly 46 is immobilized to permit flow of capture or detection entities. In an embodiment, the capture molecule assembly is immobilized on a surface or in a porous material.

The supramolecular structure 40 and/or the detector molecule assembly 46 may include a DNA origami. In some embodiments, the subcomponents of the core structure 13 of the supramolecular structure 40 and/or the detector molecule assembly 46 comprises a DNA origami as well as one or more extending nucleic acid strands. In some embodiments, the core structure 13 of the supramolecular structure 40 and/or the detector molecule assembly 46 comprises a scaffolded DNA origami, wherein a circular ssDNA molecule, called “scaffold” strand, is folded into a predefined 2D or 3D shape by interacting with 2 or more short ssDNA, called “staple” strands, which interact with specific sub-sections of the ssDNA “scaffold” strand.

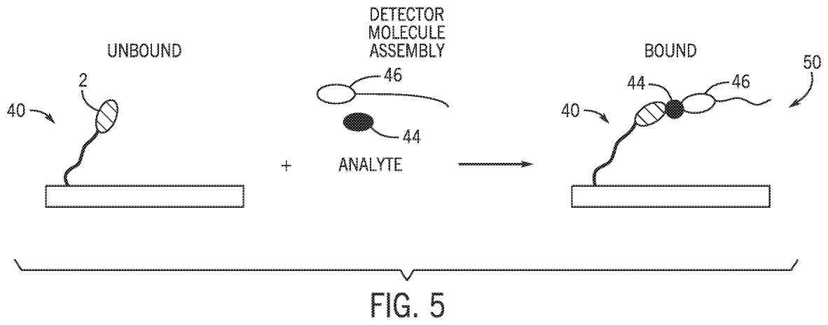

As described herein, in some embodiments, one or more supramolecular structures enable the detection of one or more analyte molecules in a sample. As shown in a schematic illustration of FIG. 5, the supramolecular structure 40, with an associated capture molecule, is exposed to an analyte molecule 44 and a detector molecule assembly 46. When the individual analyte molecule 44 and individual capture molecule 2 have binding specificity for one another, the analyte molecule 44 associates with the capture molecule 2. In turn, the detector molecule assembly 46, which has binding specificity for the analyte molecule 44, associates with the analyte molecule 44 to create a bound detection structure 50.

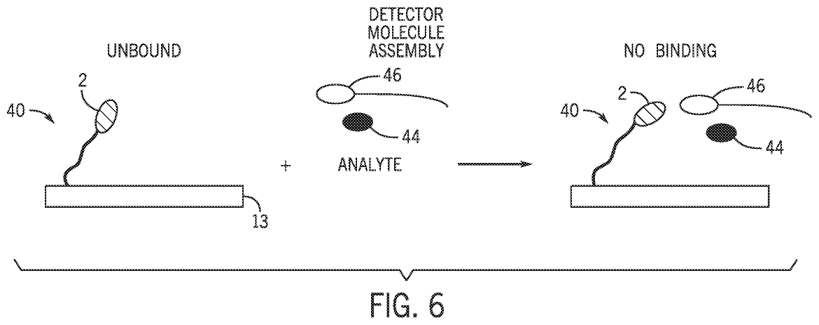

As shown in FIG. 6, analyte molecules 44 with no binding specificity for the capture molecule 2 do not associate with the supramolecular structure 40. In turn, the detector molecule assembly 46 also does not associate with the supramolecular structure 40, and no bound detection structure 50 is created. For substrates that include an immobilized array of supramolecular structures 40, each individual site may have a binding reaction to create the bound detection structure 50 when the sample includes an appropriate analyte molecule 44 with binding specificity for the capture molecule 2

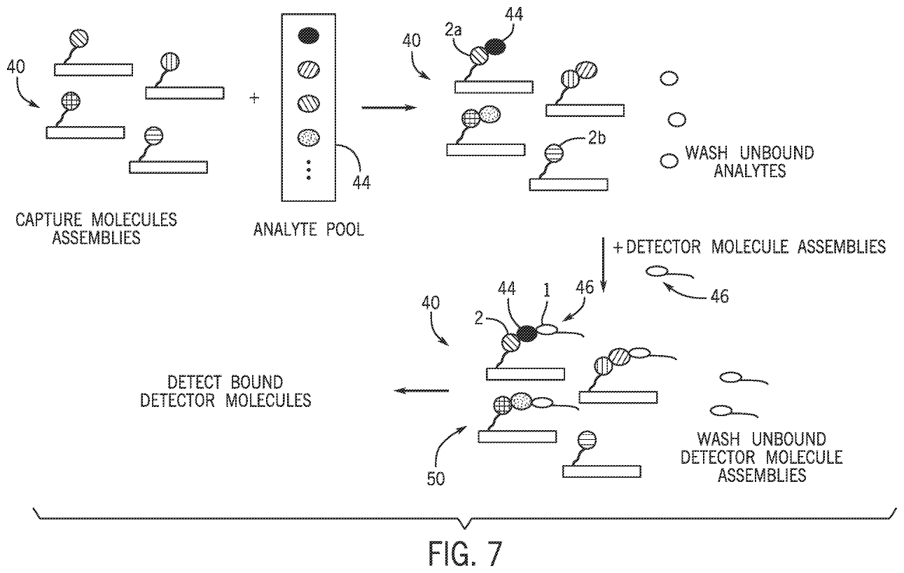

While the depicted embodiment shows the detector molecule assembly 46 and the analyte molecule 44 contacted with the supramolecular structure 40 in one step, it should be understood that the analyte molecule 44 and detector molecule assembly 46 may be added in separate steps as provided herein and such that any unbound analyte molecule 44 is removed before addition of the detector molecule assembly 46. FIG. 7 shows an example method workflow in which a pool of analyte molecules 44 is added to a group or array of capture molecule assemblies implemented as supramolecular structures 40. The analyte molecules 44 may represent different analytes present in a sample, such that different analyte molecules 44 in the pool have different degrees of binding specificity to the array of available capture molecules 2.

The reaction conditions permit binding of the analyte molecules 44 to specific capture molecules 2. As provided herein, binding specificity may refer to an interaction between the analyte molecule 44 and the capture molecule 2 that remains intact under the reaction conditions and after washing or removal steps for unbound reagents. Binding specificity may include formation of a covalent or non-covalent bonds, ionic bonds, dipole interactions, hydrophilic or hydrophobic interactions, complementary nucleic acid binding, etc. Specific binding may refer to binding to an analyte molecule 44 that binds only to a particular capture molecule 2 and not to other capture molecules 2. Thus, certain capture molecules 2 of the array bind to analyte molecules 44 (e.g., the capture molecule 2a) while other capture molecules have no available binding partners in a given sample (e.g., capture molecule 2b) and, therefore, do not bind to any analyte molecule 44 with specificity. Any unbound analytes can be removed from the capture molecule assemblies, which are immobilized as provided herein.

The detector molecule 1 and the analyte molecule 44 may also have binding specificity to one another. The array is subsequently contacted with detector molecule assemblies 46, which may all be the same or different, as disclosed in various embodiments. Any unbound detector molecule assemblies 46 are removed, e.g., by washing. After these workflow steps, various bound detector structures 50 remain on the array, each bound to respective analytes and detector molecule assemblies 46 and may be subjected to various detection protocols to associate the analyte to a particular supramolecular structure identity, which in turn is associated with a known capture molecule 2. Thus, detection permits characterization of analyte-capture molecule binding.

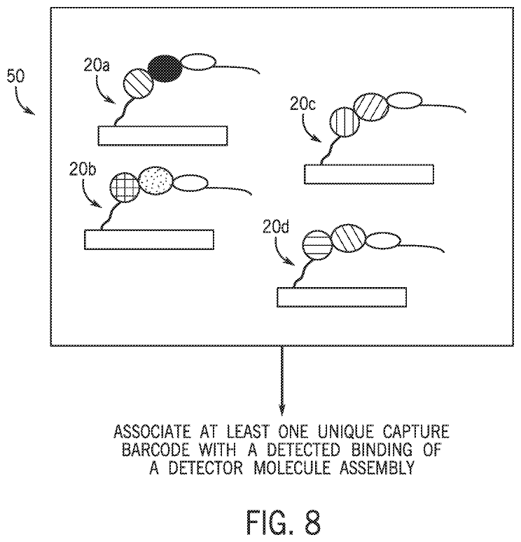

FIG. 8 shows an example detection step in which different unique capture barcodes (illustrated as capture barcodes 20a, 20b, 20c, and 20d) of the bound detector structures 50 are assessed to associate a particular capture barcode with a binding event. In some embodiments, the supramolecular structure converts information about the presence of a given analyte molecule in a sample to a DNA signal. In some embodiments, the DNA signal corresponds to sequence data for a capture barcode and/or detector barcode, wherein the capture molecule and detector molecule are simultaneously linked to (e.g., bound to) the analyte molecule (e.g., sandwich formation).

In some embodiments, detecting the presence of an analyte molecule, as described herein, comprises controllably releasing a single, or multiple, unique nucleic acid molecules into the solution to be used to identify as well as quantify properties of the analyte molecule from the sample. In some embodiments, said unique nucleic acid molecules are provided by capture barcodes 20 of the respective supramolecular structures. In some embodiments, detecting the presence of an analyte molecule, as described herein, comprises creating an optical or electrical signal connected to the state change that can be counted to quantify the concentration of the analyte molecule in solution.

In some embodiments, a plurality of analyte molecules are simultaneously detected in a sample through multiplexing, wherein a plurality of supramolecular structures provide a plurality of signals (e.g., detector barcode, capture barcode) for sequencing and analyte identification. In some embodiments, methods described herein for detecting analytes in a sample provide a high-throughput and high-multiplexing capability by using a plurality of supramolecular structures. In some embodiments, the high-throughput and high-multiplexing capability provides high accuracy for analyte molecule detection and quantification. In some embodiments, methods described herein for detecting analytes in a sample are configured to characterize and/or identify biopolymers, including proteins molecules, quickly and at high sensitivity and reproducibility. In some embodiments, the plurality of supramolecular structures are configured to limit cross-reactivity associated errors. In some embodiments, such cross-reactivity associated errors comprise capture and/or detector molecules of a supramolecular structure interacting with capture and/or detector molecules of another supramolecular structure (e.g., intermolecular interactions). In some embodiments, each core structure of the plurality of supramolecular structures is identical to one another. In some embodiments, the structural, chemical, and physical property of each supramolecular structure is explicitly designed. In some embodiments, identical core structures have a prescribed shape, size, molecular weight, prescribed number of capture and detector molecules, predetermined distance between corresponding capture and detector molecules (as described herein), prescribed stoichiometry between corresponding capture and detector molecules, or combinations thereof, so as to limit the cross-reactivity between supramolecular structures. In some embodiments, the molecular weight of every core structure is identical and precise up to the purity of the core molecules. In some embodiments, each core structure has at least one capture molecule.

In some embodiments, the plurality of supramolecular structures might share structural similarities due to certain subcomponents being the same, however the interaction between an analyte molecule from the sample and supramolecular structure is defined by the corresponding capture molecule and detector molecule. In some embodiments, each bound detector and capture molecules on a given bound detection structure 50 may specifically interact with a particular analyte molecule in the sample. In some embodiments, each supramolecular structure comprises unique DNA barcodes corresponding to the associated capture molecule. In some embodiments, a capture molecules is designed to interact with more than one analyte molecule in the sample.

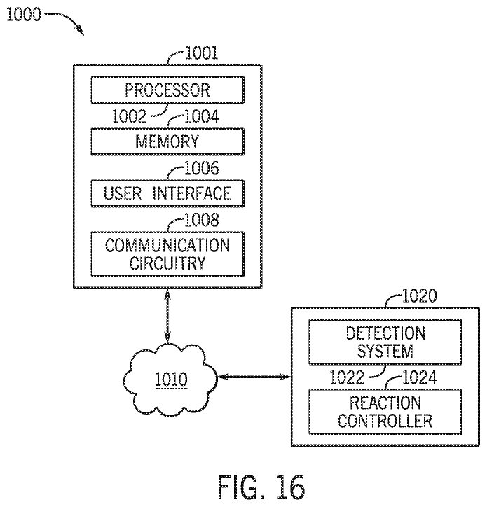

As provided herein, the capture barcode 20 can be used to uniquely identify individual supramolecular structures 40. In turn, each supramolecular structure 40 is assembled so that the capture molecule 2 may be associated with the capture barcode 20, e.g., stored in a lookup table of an analyte detection system (see FIG. 16). Thus, when the capture barcode 20 is identified, the identity of the capture molecule 2 is also accessible.

In some embodiments, each supramolecular structure is configured for single-molecule sensitivity to ensure the highest possible dynamic range needed to quantitatively capture the wide range of molecular concentrations within a typical complex biological sample. In some embodiments, the plurality of supramolecular structures limit or eliminate the manipulation of the sample needed to reduce non-specific interaction as well as any user induced errors.

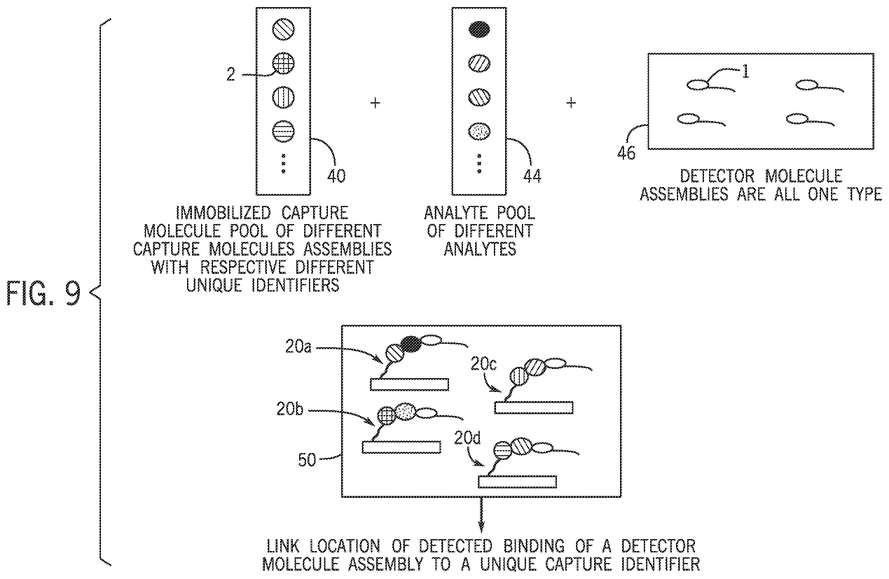

FIG. 9 shows an example analyte detection technique in which different the locations of supramolecular structures 40 with respective different capture molecules 2, together with unique capture barcode information, can be used to characterize analyte binding. A sample including a pool of different analyte molecules 44 is contacted with immobilized supramolecular structures 40 with respective different capture molecules 2. Analyte molecules 44 and capture molecules 2 with binding specificity for one another are contacted under conditions to allow the interaction to occur. Detector molecule assemblies 46 are permitted to bind to analyte molecules 44 that are associated with the supramolecular structures. The bound detector structures can be characterized based on 1) the capture barcode 20 and 2) a signal generated by a bound detector molecule assembly that corresponds to a location map of a particular capture barcode 20. In an embodiment, the locations of the supramolecular structures 40, together with capture barcode information, can be determined before analyte binding. That is, the array may be provided pre-mapped, or the mapping can be a separate step. The mapping may include a step of detecting the capture barcode as generally provided herein, such as detecting a unique optical, electrical, and/or magnetic pattern. In an embodiment, the detection includes sequencing a nucleotide sequence of the capture barcode. In an embodiment, the detection includes amplification and quantitation of the amplified product, e.g., detection of a signal associated with a probe via qPCR.

Serology tests look for antibodies in a patient's blood to identify a past infection with a pathogen. In one example, COVID-19 serology assays detect the presence of IgG or IgM antibodies against spike protein or nucleocapsid. The capture molecules 2 pull down antibody analytes 44 in the patient sample, which are also bound by detector molecule assemblies 46. In an embodiment, the detector molecule assemblies 46 may be all of a same type and/or all have a same detector molecule 1. The detector molecule 1 can be an anti-human antibody that binds to any human antibody, regardless of the antibody-antigen specificity. Thus, the detector molecule 1 is capable of binding a range of different analytes 44 associated with respective antigen capture molecules 2. The analytes 44 that represent a positive binding event via a detectable signal from the detector molecule assemblies 46 can be linked to a particular supramolecular structure 40 based on the particular barcode 20 to identify the positive antibody result. The disclosed techniques may be used to create an assay for one or more infectious diseases, such as COVID-19, Influenza, RSV, and Pneumonia. In an embodiment, the capture molecules 2 include a pool of different antigens of different infectious diseases and respectively associated with different supramolecular structures 40. Additionally or alternatively, the assay may include multiple isoforms and multiple potential antigens of an infectious disease as well as the antigens from other respiratory pathogens including but not limited to Influenza, RSV, and Pneumonia. In an embodiment, the assay may permit differentiation between natural immunity and gained immunity from a vaccine through the specific addition of vaccine protein targets. The assay may also include the differentiation between IgG, IgM and IgA specificity. This assay can be updated or modified seasonally as new infections arises to appropriately interrogate the current pathogen climate. The improvement made by this assay to the current workflow will give greater insight to the patient's humoral immune system, as well as help inform vaccine development. For example, a patient's antibody response or circulating antibody population can be assessed for binding to various candidate antigens.

The detector molecule assemblies 46 may be all of a same type or may be detected using a same detection modality. The detected signal may not include any unique barcode information. In one example, the detector tail may include a reactive molecule that generates the signal. In an embodiment, the detector molecule assemblies 46 are detected based on enzyme conversion of a substrate to an optically detectable product. The optical detection is associated with a spot on the array of supramolecular structures 40. In an embodiment, the detector molecule 1 of each of the detector molecule assemblies 46 may be all of a same type and/or have a same binding specificity. In one example, the analyte molecules 44 detected are all human antibodies, and the detector molecule is an anti-human antibody with general binding specificity to a wide range of human antibodies, regardless of antigen specificity. Other embodiments are also contemplated. For example, the analyte molecules 44 may undergo a tagging or processing step to add a tag (e.g., biotin) that permits binding to streptavidin detector molecules 1. In the depicted embodiment, the step of providing the detector molecule assemblies 46 may be less complex, because the pool of detector molecule assemblies 46 is not diverse, and the detector molecules 1 and associated tail or linkers may also be all of the same type. Further, the detection step may also be less complex, because no sequence or barcode information from the detector side is obtained. Thus, in an embodiment, the unique identification information is the capture barcode 20 that is used together with location information for each capture barcode and a detector-generated signal location. Correlation of the detector signal with location of particular barcodes is used to characterize analyte binding. It should be understood that the method of analyte detection FIG. 9 may also be performed using a diverse pool of detector molecule assemblies having unique barcodes as well as reactive molecules that generate the detector signal.

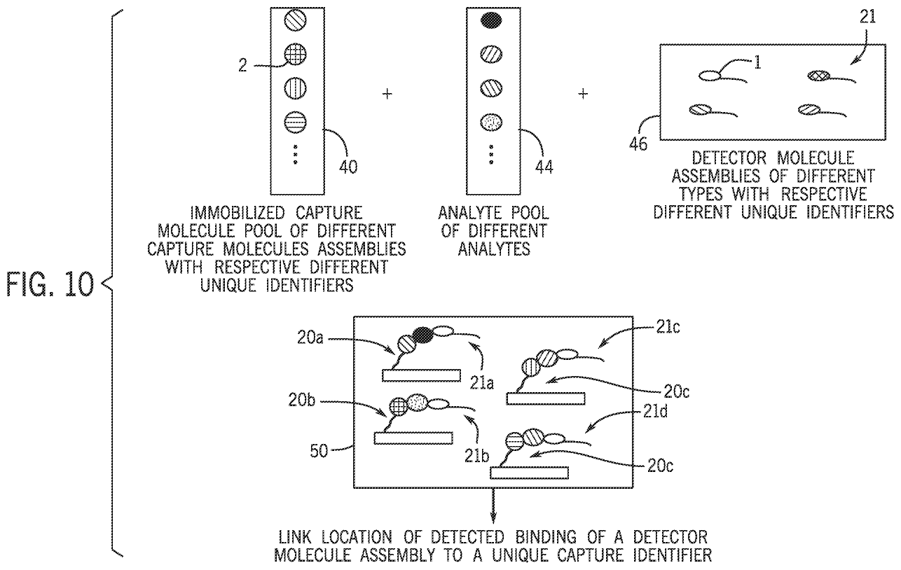

FIG. 10 shows an embodiment that is similar to the analyte detection method of FIG. 9, but in which the pool of detector molecule assemblies 46 is diverse. The diverse pool of detector molecule assemblies 46 carries different respective detector molecules 1 and detector barcodes 21 (shown as detector barcode 21a, 21b, 21c, and 21d). The bound detector structures 50 include both a unique capture barcode 20 associated with the capture molecule 2 specific for a particular analyte molecule 44 as well as a unique detector barcode 21 specific for the analyte molecule 44. One or both of the unique capture barcode 20 or the unique detector barcode 21 can be assessed to characterize analyte binding. Detection of the unique detector barcode 21 may be performed using techniques discussed with reference to the unique capture barcode 20, including optical, electrical, and/or magnetic detection. Detection may include generating sequence data or amplification data of the unique detector barcode 21.

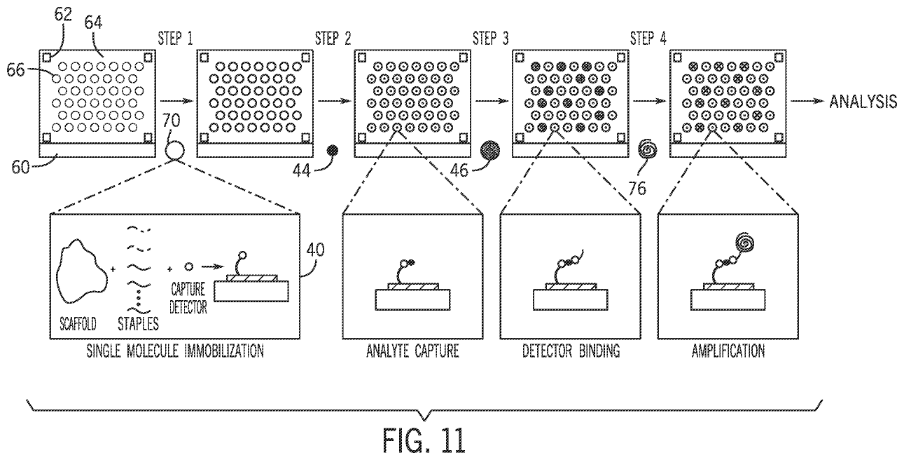

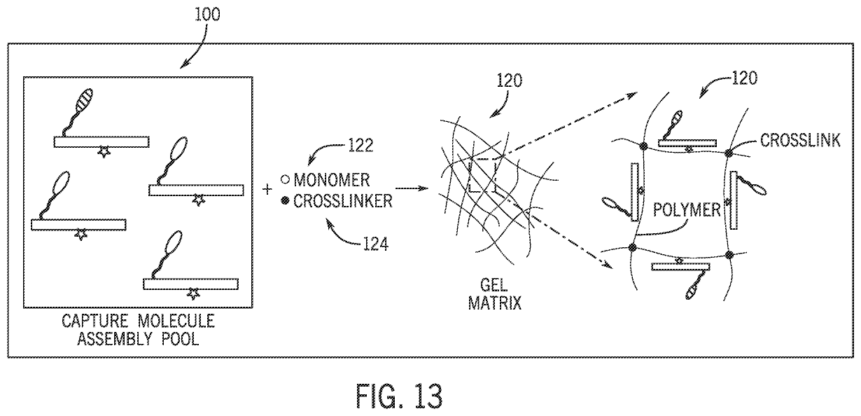

In some embodiments, the sample, comprising one or more analytes is contacted with the one or more supramolecular structures 40. In some embodiments, as described herein, the plurality of supramolecular structures are provided as being attached to one or more solid substrates. FIGS. 11-14 provide examples of supramolecular structures attached to a patterned solid substrate. FIG. 15 shows an example of supramolecular structures incorporated into a porous hydrogel matrix. The disclosed techniques may be performed in conjunction with a patterned substrate including binding sites distributed on or in the binding site. In an embodiment, each binding site accommodates a single supramolecular structure 40 with differential chemistry. The patterned substrate may be fabricated through lithography processes. Further, embodiments of the disclosed techniques may include one or more regeneration steps that remove a bound or “used” bound detector structure 50 from the substrate to incorporate a new supramolecular structure 40.

FIG. 11 provides an exemplary illustration of a method for detecting analyte molecules in a sample using a surface based assay that uses supramolecular structures 40, as described herein, for single-molecule counting of analytes in the sample (i.e. detecting analyte molecules in the sample at a single molecule resolution). In some embodiments, the supramolecular structures comprise a core structure comprising a DNA origami core. In some embodiments, a planar substrate 60 is provided comprising (a) Fiduciary markers 62 that serves as a reference coordinates for all the features on the substrate 60; (b) A defined set of micropatterned binding sites 66 where individual core structures (e.g., DNA origami) may be immobilized; (c) background passivation 64 that minimizes or prevents interaction between the surface of the substrate 60 and the supramolecular structure (including capture molecules, core structure molecules). In some embodiments, the fiduciary markers comprise geometric features defined on a surface to be used as reference features for other features on the substrate. In some embodiments, the fiduciary markers 62 are coated with a polymer or self-assembled monolayer that does not interact with a core structure or other molecules of the supramolecular structure (e.g., DNA origami). In some embodiments, the background passivation 64 minimizes or prevents interaction between the surface of the substrate 60 and analyte molecules of the sample. In some embodiments, the planar substrate 60 comprises optical or electrical devices like FET, ring resonators, photonic crystals or microelectrode, to be defined prior to the formation of the binding sites 66. In some embodiments, the binding sites 66 are micropatterned on the planar substrate 60. In some embodiments, the binding sites 66 on the surface are in a periodic pattern. In some embodiments, the binding sites 66 on the surface are in a non-periodic pattern (e.g., random). In some embodiments, a minimum distance is specified between any two binding sites 66. In some embodiments, the minimum distance between any two binding sites 66 is at least about 200 nm. In some embodiments, the minimum distance between any two binding sites 66 is from at least about 40 nm to about 5000 nm. In some embodiments, the geometric shape of the binding sites 66 comprises a circle, square, triangle or other polygon shapes. In some embodiments, the chemical groups that are used for passivation 64 comprise neutrally charged molecules like a Tri-methyl silyl (TMS), an uncharged polymer like PEG a zwitterionic polymer like, or combinations thereof. In some embodiments, the chemical group used to define the binding site 66 comprises a silanol group, carboxyl group, thiol, other groups, or combinations thereof.

In some embodiments, a single supramolecular structure 40 is attached to a respective binding site 66 (Step 1). Reference character 70 provides a depiction of the components of the supramolecular structure 40, individually and as assembled and arranged on the planar substrate (components are as described herein, e.g., FIGS. 1-4). In some embodiments, the supramolecular structure 40 comprises a core structure 13 comprising a DNA origami, wherein the supramolecular structures 40 is attached onto each of the binding sites using DNA origami placement technique (step 1). In some embodiments, the supramolecular structure 40 is assembled prior to being attached to a respective binding site 66. In some embodiments, the DNA origami comprises a unique shape and dimension, so as to facilitate binding to a binding site using the DNA origami placement technique. In some embodiments, DNA origami placement comprises a directed self-assembly technique for organizing individual DNA origami (e.g., a core structure) on a surface (e.g., micropatterned surface). In some embodiments, alternatively to the DNA origami placement, a reactive group of the supramolecular nanostructure 40 is bound to a DNA origami that has been pre-organized on the binding site. In some embodiments, both of these methods for binding a supramolecular nanostructure to a corresponding binding site rely on the ability to organize one or more molecules on a micropatterned binding site using the DNA origami placement technique. In some embodiments, the planar substrate could be stored for a significant period after this step, in a clean environment.

With continued reference to FIG. 11, in some embodiments, a sample (as described herein) comprising analyte molecules is contacted with the planar substrate (step 2) in an analyte capture step. In some embodiments, the sample is contacted with the planar substrate using a flow-cell. In some embodiments, the sample is incubated on the planar substrate with the supramolecular structures attached to the binding sites 66. In some embodiments, the incubation period may be from about 30 seconds to about 24 hours. In some embodiments, the incubation period may be from about 30 seconds to about 1 minute, from about 1 minute to about 5 minutes, from about 5 minutes to about 30 minutes, from about 30 minutes to about 1 hr, from about 1 hr to about 5 hours, from about 5 hours to about 12 hours, from about 12 hours to about 24 hours, from about 24 hours to about 48 hours.

In some embodiments, the analyte molecules 44, in the sample, interact with the supramolecular structures 40 on the planar surface 60. In some embodiments, a single copy of a specific analyte molecule 44 binds to a capture molecule. With continued reference to FIG. 11, at step 3, detector molecule assemblies 46 are contacted with the captured analytes. The detector molecular assemblies are detected to generate a detectable signal at step 4. For example, the detector barcode 21 is used as a binding site for a signaling element 76 that is contacted with the bound detector molecule assemblies 46. In some embodiments, the signaling element 76 comprises a fluorescent molecule or microbead, a fluorescent polymer, highly charged nanoparticles or polymer. In some embodiments, one or more signaling elements 76 are allowed to interact with the supramolecular structures on the planar structure. In some embodiments, the signaling elements 76 are introduced into the flow-cell containing the planar substrates. In some embodiments, the detector barcode is amplified, as shown in the depicted embodiment. For example, the detector barcode is used as a polymerization initiator for growth of highly fluorescent polymer in a process such as rolling circle amplification or hybridization chain reaction. In some embodiments, the detectable signal as described with step 4 leads to a surface in which every individual analyte capture event leads to a signaling element 76 being present at the location of the respective analyte (as linked with the capture and detector molecules).

In some embodiments, the signaling element 76 is optically active and can be measured using a microscope or integrated optically sensor within the planar substrate 60. In some embodiments, the signaling element is electrically active and may be measured using an integrated electrical sensor. In some embodiments, the signaling element 76 is magnetically active and may be measured using an integrated magnetic sensor. In some embodiments, each signal event is associated with the capture of the same type of analyte molecule (a single copy of the same type of analyte molecule), determined by the corresponding detector and capture molecule, thus counting the number of locations where the signaling element 76 is present gives the quantification of the analyte molecule in the sample

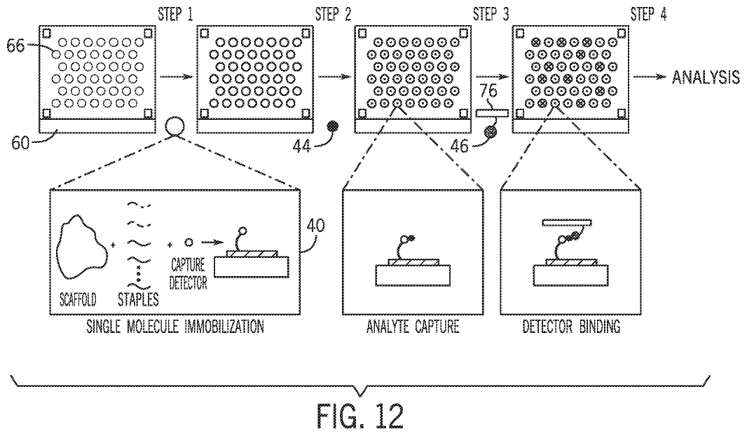

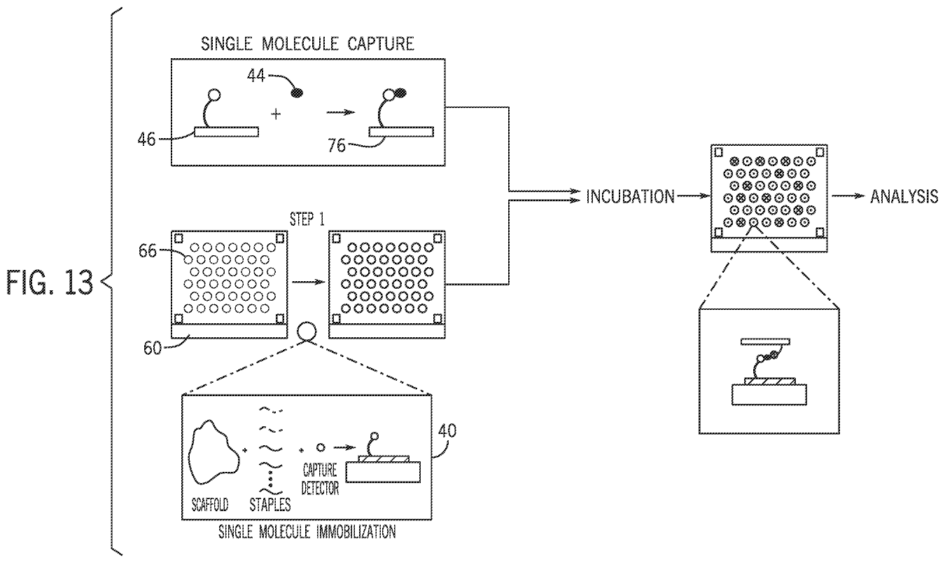

FIG. 12 shows an arrangement having a similar planar substrate 60 with binding sites 66 as in FIG. 11. The planar substrate 60 has assembled supramolecular structures 40 immobilized at respective binding sites 66 (step 1). After analyte capture (step 2), detector molecule assemblies are contacted with the captured analyte molecules 44. Here, each detector molecule assembly 46 includes a bound core structure (e.g., core structure 13, see FIG. 1), such as DNA origami, that operates as an integral signaling element 76. FIG. 13 shows an arrangement in which the planar substrate 60 (which may be formed as described with respect to FIG. 11) has assembled supramolecular structures 40 immobilized at respective binding sites 66 (step 1). A separate process associates analytes 44 with respective detector molecule assemblies 46. In an embodiment, each detector molecule assembly 46 carries a signal element 76, such as a supramolecular core structure. The associated analytes and detector molecule assemblies 46 are incubated with the planar substrate 60 and immobilized supramolecular structures 40. The associated analytes and detector molecule assemblies 46 bind to supramolecular structures having capture molecules with binding specificity for the analyte, and the bound analytes can be characterized e.g., via detecting signals generated from a signaling element (which may be part of the detector molecule assembly 46 or which may be added after association of the analytes and detector molecule assemblies 46 with the capture molecules).

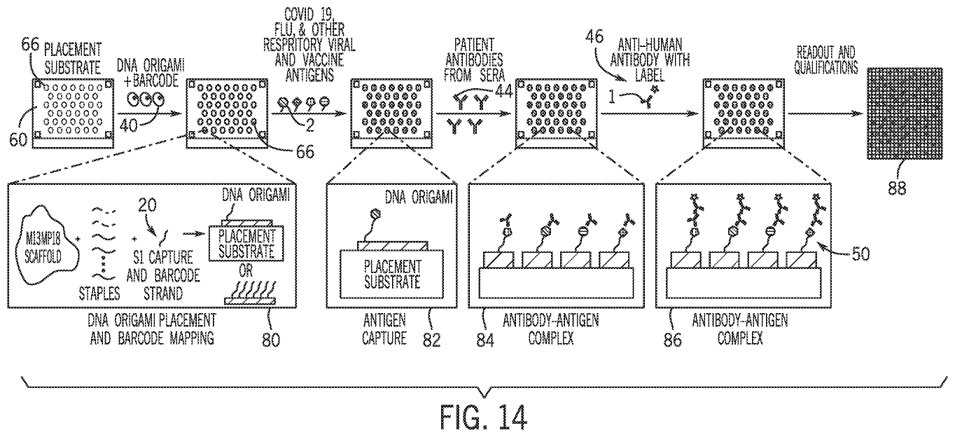

In FIG. 14, a patterned substrate 60 with multiple binding sites 66 can be functionalized with or linked to individual supramolecular structures 40. In the depicted embodiment, the supramolecular structure 40 is assembled at step 80 and then arranged on the substrate 60 before an antigen capture molecule 2 is linked. For example, a DNA origami containing a specific barcode and capture strand specific to a particular antigen is flowed over the substrate 60 and placed in a single molecule array, e.g., on DNA binding features. Antigens are conjugated to include a complementary strand, shown as capture molecules 2, which can anneal to the DNA origami barcode 20 of the supramolecular structures at step 82. Alternatively, the capture molecule is associated with the supramolecular structure 40 during assembly (step 80) and is applied to the substrate 60 together with the supramolecular structure 40. The barcode 20 of each supramolecular structure is read out either via sequencing, amplification, or via hybridization assay. A map of antigen capture molecules 2 with the spatial locations on the substrate 60 can be obtained prior to performing the assay and performed as quality control of substrate 60. The map can be stored in an analyte detection system (see FIG. 16) as provided herein and used to generate a report of positive binding events to provide diagnostic information.

Appropriate blocking conditions added and a sample including patient antibodies (e.g., serum) is then applied to the substrate 60 and allowed to incubate to allow antibody analytes 44 in the sample to form antibody-antigen complexes at step 84. The sample is then washed off and a detector molecular assembly 46 including a detector molecule 1 that is a secondary anti-human antibody and a label for detection is added at step 86. The detection (step 88) of bound detector structures 50 can be based on detection of the antibody label, which can be DNA-based for amplification either through rolling circle amplification, or hybrid chain reaction; alternatively, the label can be a DNA nanoparticle, or fluorescent polymer.

In an embodiment, the assay includes antigens of one or more of Adenovirus, Coronavirus 229E, Coronavirus HKU1, Coronavirus B.1.1.7, Coronavirus B.1.351, Coronavirus P.1 Coronavirus NL63, Coronavirus OC43, Human metapneumovirus, Human rhinovirus/enterovirus, Influenza A, subtypes 2009H1N1, H1, H3, Influenza B, Parainfluenza virus types 1, 2, 3 and 4, Respiratory Syncytial Virus, Chlamydophila pneumoniae, Mycoplasma pneumoniae, and Bordetella Pertussis. In addition, the assay may include vaccine targets for the seasonal flu, as well as the covid vaccines targets for one or more commercial vaccines (Moderna, Pfizer, Astra Zeneca, Novavax, and Johnson and Johnson). The addition of these vaccine targets allows for the classification of the immunity to determine whether a patient has immunity from natural infection and/or gained immunity from the vaccine. Multiple antigens for each pathogen may be present to define specificity of the immunity.

Detection may include anti-species for specific antibodies for IgG, IgM and/or IgA determination. Specific detection of subtype aids in further understanding maturity of immunity.

FIG. 15 provides an exemplary embodiment for forming a hydrogel matrix 100, wherein in addition to combining one or more monomers 122 and one or more crosslinking molecules 124 to form a hydrogel, one or more supramolecular structures 40 are introduced. In some embodiments, the one or more supramolecular structures 40 co-polymerizes with the hydrogel matrix, forming the matrix 120. In some embodiments, each respective anchor molecule 18 of the one or more supramolecular structures 40 co-polymerizes with the hydrogel matrix 120. In some embodiments, the one or more monomers 122 comprise an acrylamide. In some embodiments, the one or more cross-linkers comprise a bis-acrylamide.

Embodiments of the present disclosure include one or more computer-implemented detection systems configured to perform certain methods of the disclosed embodiments. FIG. 16 shows an analyte detection system 1000 that includes a controller 1001. The controller 1001 includes processor 1002 and a memory 1004 storing instructions configured to be executed by the processor 1002. The controller 1001 includes a user interface 1006 and communication circuitry, e.g., to facilitate communication over the internet 1010 and/or over a wireless or wired network. The user interface 1006 facilitates user interaction with characterized analyte detection results as provided herein.

The processor 1002 is programmed to receive analyte detection data and characterize the detected analytes. In one embodiment, the processor generates a report of detected analytes in a sample after incubation with an array of supramolecular structures and detection of detector molecule assemblies. The report may include data of generated optical signals at various binding sites that corresponds to a detected analyte binding event. The report may include processed data, such as a list of detected analytes or positive/negative binding results. The report may include a list of available capture molecules of an array that is indicative of the analyte detection capabilities.

The system 1000 also includes an analyte detector 1020 that operates to detect analyte binding via detection of one or more components of the supramolecular structure. The analyte detector 1020 includes a detection system having one or more sensors 1022. The analyte detector 1020 may also include a reaction controller 1024 that controls sample incubation and appropriate release of reaction reagents and detector molecule assemblies at appropriate time points. The sensor 1022 may be one or more of an optical sensor (e.g., a fluorescent sensor, an infrared sensor), an image sensor, an electrical sensor, or a magnetic sensor. In an embodiment, the sensor 102 is a metal-oxide semiconductor image sensor device.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for detecting an analyte molecule present in a sample, the method comprising:
providing an array of supramolecular structures immobilized on a substrate or in a porous matrix, a supramolecular structure of the array comprising:
a core structure comprising a plurality of core molecules; and
a capture molecule linked to the core structure via a capture barcode contacting the sample with the array such that the analyte molecule binds to the capture molecule;
contacting the analyte molecule bound to the capture molecule with detector molecule assemblies such that a detector molecule of an individual detector molecule assembly binds to the analyte molecule to form a bound detection structure;
detecting the analyte molecule based on a signal generated by the individual detector molecule assembly of the bound detection structure; and
associating the detected analyte molecule with the supramolecular structure based on an identity of the capture barcode.

2. The method of claim 1, further comprising removing additional analyte molecules in the sample not bound to supramolecular structures of the array after contacting the sample with the array.

3. The method of claim 1, further comprising removing detector molecule assemblies not bound to supramolecular structures of the array after forming the bound detection structure.

4. The method of claim 1, wherein the analyte molecule comprises a protein, a peptide, a peptide fragment, a lipid, a DNA, a RNA, an organic molecule, an inorganic molecule, complexes thereof, or any combinations thereof.

5. The method of claim 1, wherein the supramolecular structure of the array is a nanostructure.

6. The method of claim 5, wherein the core structure is a nanostructure.

7. The method claim 1, wherein a plurality of core molecules of the core structure are arranged into a pre-defined shape and/or have a prescribed molecular weight.

8. The method claim of 7, wherein the pre-defined shape is configured to limit or prevent cross-reactivity with another supramolecular structure.

9. The method of claim 1, wherein the core structure comprises a scaffolded deoxyribonucleic acid (DNA) origami, a scaffolded ribonucleic acid (RNA) origami, a scaffolded hybrid DNA:RNA origami, a single-stranded DNA tile structure, a multi-stranded DNA tile structure, a single-stranded RNA origami, a multi-stranded RNA tile structure, hierarchically composed DNA or RNA origami with multiple scaffolds, a peptide structure, or combinations thereof.

10. The method of claim 1, wherein the capture barcode comprises a first capture linker, a second capture linker, and a capture bridge disposed between the first and second capture linkers, wherein the first capture linker is bound to a first core linker that is bound to the first location on the core structure, wherein the capture molecule and the second capture linker are linked together through binding to a third capture linker.

11. The method of claim 1, wherein the capture molecule of the individual supramolecular structure has binding specificity for the analyte molecule and wherein a second capture molecule of at least one other supramolecular structure of the array does not have binding specificity for the analyte molecule.

12. The method of claim 1, wherein respective capture molecules of the supramolecular structures of the array have different binding specificity to respective different analyte molecules.

13. The method of claim 1, wherein the detector molecule of the detector molecule assembly has binding specificity for the analyte molecule and wherein other detector molecules of the detector molecule assemblies do not have binding specificity for the analyte molecule.

14. The method of claim 1, wherein detector molecules of the detector molecule assemblies have different binding specificity to respective different analyte molecules.

15. The method of claim 1, wherein detector molecules of the detector molecule assemblies have binding specificity for any analyte molecule bound to the capture molecules of the supramolecular structures.

16. The method of claim 1, wherein detector molecules of the detector molecule assemblies have no binding specificity for the supramolecular structures.

17. The method of claim 1, comprising determining a nucleic acid sequence of the capture barcode to determine the identity of the supramolecular structure.

18. The method of claim 17, comprising associating the determined nucleic acid sequence of the supramolecular structure to a location on the array.

19. The method of claim 18, wherein associating the detected analyte molecule with the supramolecular structure based on the identity of the capture barcode comprises associating a location of the signal with the location on the array of the nucleic acid sequence.

20. The method of claim 18, wherein the signal is an optically, magnetically, and/or electrically detectable signal generated by a reactive molecule of the detector molecule assembly.

21. The method of claim 18, wherein the signal is a sequence of a detector barcode of the detector molecule assembly.

22. An array for detecting one or more analyte molecules in a sample, comprising:
a substrate;
a plurality of supramolecular structures immobilized on the substrate, wherein an individual supramolecular structure of the plurality of supramolecular structures comprises:
(a) a core structure comprising a plurality of core molecules, wherein the core structure is coupled to substrate or is linked to the substrate by an anchor molecule,
(b) a capture barcode coupled directly or indirectly to the core structure at a first end of the capture barcode, the capture barcode extending generally away from the substrate; and
(c) a capture molecule coupled to a capture barcode at a second end of the capture barcode, the capture molecule being configured to bind to an analyte molecule.

23. The substrate of claim 22, wherein each core structure of the plurality of supramolecular structures is identical to each other.

24. The substrate of claim 22, wherein each supramolecular structure has a unique capture barcode.

25. The substrate of claim 22, wherein the substrate comprises a solid support or a porous matrix.

26. The substrate of claim 22, wherein each supramolecular structure is a nanostructure.

27. The substrate of claim 22, wherein each core structure is a nanostructure.

28. The substrate of claim 22, wherein the plurality of core molecules for each core structure are arranged into a pre-defined shape and/or have a prescribed molecular weight.

29. The substrate of claim 22, wherein the core structure is directly coupled to the substrate.

30. The substrate of claim 22, comprising a plurality of analyte molecules bound to respective supramolecular structures of the plurality of supramolecular structures.

31. The substrate of claim 30, comprising a plurality of detector molecule assemblies bound to respective analyte molecules of the plurality of analyte molecules.

32. The substrate of claim 31, wherein each detector molecule assembly comprises a detector molecule coupled to a detector barcode comprising one or more linkers.

33. The substrate of claim 32, wherein each detector molecule assembly comprises a core structure coupled to the detector molecule by the detector barcode.

34. The substrate of claim 22, wherein the substrate comprises a porous matrix.

35. The substrate of claim 34, wherein the porous matrix comprises a hydrogel.

36. The substrate of claim 22, wherein the substrate comprises a planar substrate.

37. The substrate of claim 22, wherein the individual supramolecular structure comprises only one capture molecule.

38. A substrate for detecting one or more analyte molecules in a sample, the substrate comprising:
a patterned substrate comprising a plurality of binding sites spaced apart from one another; and
a single supramolecular structure associated with each binding site of the plurality of binding sites, the supramolecular structure comprising:
a core structure comprising a plurality of core molecules, wherein the core structure is coupled to substrate or is linked to the substrate by an anchor molecule,
a capture barcode coupled directly or indirectly to the core structure at a first end of the capture barcode, the capture barcode extending generally away from the substrate; and
a capture molecule coupled to the capture barcode at a second end of the capture barcode, the capture molecule being configured to bind to an analyte molecule.

39. The substrate of claim 38, comprising one or more fiducial markers disposed on or in the patterned substrate, wherein the one or more fiducial markers are detectable by a detection system to provide location information for the supramolecular structure.

40. The substrate of claim 38, comprising passivated regions on the patterned substrate that separate the plurality of binding sites.

41. The substrate of claim 38, wherein each binding site of the plurality of binding sites is sized to accommodate the single supramolecular structure.

42. An array for detecting one or more analyte molecules in a sample, the array comprising:
a patterned substrate comprising a plurality of binding sites; and
a supramolecular structure associated with each binding site of the plurality of binding sites, the supramolecular structure comprising:
a core structure comprising a plurality of core molecules, wherein the core structure is coupled to substrate or is linked to the substrate by an anchor molecule,
a capture barcode coupled directly or indirectly to the core structure at a first end of the capture barcode, the capture barcode extending generally away from the substrate; and
a capture molecule coupled to a capture barcode at a second end of the capture barcode, the capture molecule being configured to bind to an analyte molecule.
